(19) United States
(12) Patent Application Publication
(10) Pub. No.: US 2007/0078411 A1
(43) Pub. Date: Apr. 5, 2007

(54) Indwelling catheter opening device

(76) Inventors: Thomas A. Osborne, Bloomington, IN (US); Julie Behen, Bloomington, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/196,700

(22) Filed: Aug. 3, 2005

(51) Int. Cl. A61M 5/00; A61M 5/178; A61M 5/14

(52) U.S. Cl. 604/256; 604/170.01

(57) ABSTRACT

A device and method for opening a substantially flat external flange of an indwelling catheter. The device is a one-piece elongated member having configured ends formed by bending the elongated member so that one configured end is positioned slightly above the other configured end by passing the second configured end through an aperture formed inside of the member. In addition, an apparatus comprising at least the opening device and an indwelling catheter having a flat external flange is disclosed. Other embodiments of opening devices constructed and arranged to open the external flange of an indwelling catheter are also contemplated. The device is used to maintain the low profile of the external flange of the indwelling catheter and to prevent damage to the skin or body tissue surrounding the catheter flange.

### TECHNICAL FIELD

The present invention generally relates to medical devices and particularly, but not exclusively, to a device to open the flat external flange of an indwelling catheter.

### BACKGROUND

The following catheter and opening device are commonly used in the areas of gastronomy and gastrojejunostoy and cecostomy. While there is particular use in these fields, those skilled in the art recognize that the opener may be used for any catheter that is used in any application where the connection is from the outside of the patient's body to an internal cavity within the body. Some other uses may be in the biliary tree, the liver, the kidney, etc. without limitation to only these places.

Heretofore, catheters have been produced with a flat external flange. The flat external flange has prevented distress for many patients. It is emotionally stressful to have a catheter protruding out of one's body and therefore a flat external flange helps to minimize this upsetting visual. In addition, lying on the catheter is uncomfortable if the catheter is not flat and it protrudes out from the body. Also, catheters that protrude out are problematic with pediatric patients because they will not leave the catheter alone and many times end up pulling it out. Therefore, catheters have been developed with a substantially flat external flange to alleviate these problems.

As commonly occurs, however, the solution many times creates its own problems. A catheter with a very flat external flange has a very low profile making it difficult to grab the flange in order to open and close the catheter. Moreover, it is difficult to manually open the flange without disturbing the placement of the catheter. As a result, the external flange will end up protruding slightly above the surface of the skin after accessing the catheter a few times. Also, sensitive or wounded skin around the catheter may be damaged. To alleviate these problems, it is preferable that the flanges be flush with or in contact with the skin in order to provide the lowest profile possible.

Therefore, needs remain in this area of technology.

### SUMMARY

One aspect of the present invention is a device for opening the access area of a catheter. The device includes a substantially flat elongate member comprising: a first configured end portion; a second configured end portion; and a body portion between the configured end portions, the body portion defining an aperture, wherein the body portion is formed so as to position the second configured end portion above the first configured end portion by passing the second configured end portion through the aperture in the body portion.

Another aspect of the present invention is an apparatus. The apparatus includes a catheter having a substantially flat external flange; and an opening device comprising: a first configured end portion; a second configured end portion; and a body portion between the configured end portions, the body portion defining an aperture, wherein the body portion is formed so as to position the second configured end portion above the first configured end portion by passing the second configured end portion through the aperture, wherein the opening device opens the external flange of the catheter.

Yet a further aspect of the invention includes a medical apparatus for a human body. The apparatus includes a catheter comprising: an elongate body having a proximal end, a distal end and an interior lumen therethrough; an external flange having a substantially flat base, a substantially flat cover and a hinge coupling the cover to the base; and a plurality of helical coils formed in the body between the proximal and distal ends, wherein a stiffener is placed into the lumen to straighten the coils to enable insertion into the body and the coils reform upon removal of the stiffener to enable retention of the catheter in the body; and a device constructed and arranged to slide between and physically separate the base and the cover of the external flange.

Another aspect of the invention includes a method for opening a catheter having a substantially flat external flange formed having two substantially flat plates with an opening device having at least two configured portions. The method includes the steps of inserting the configured portions between the substantially flat plates; and compressing the opening device to apply forces to the substantially flat plates using the configured portions.

### BRIEF DESCRIPTION OF THE DRAWINGS

FIG. 1 is a side elevational view of a straightener and a prior art catheter having a substantially flat external flange.

FIG. 2 is an elevational view of the prior art catheter of FIG. 1.

FIG. 3 is a perspective view of one embodiment of an opening device according to the present invention.

FIG. 4A is a side elevational view of the device of FIG. 3 immediately prior to opening the catheter flange of FIG. 1.

FIG. 4B is a side elevational view illustrating the device in FIG. 3 opening the catheter flange illustrated in FIG. 1.

FIG. 5A is a side elevational view of the device of FIG. 3 immediately prior to closing the catheter flange of FIG. 1.

FIG. 5B is a side elevational view of the device of FIG. 3 closing the catheter flange of FIG. 1.

FIG. 6A is a top plan view of the device of FIG. 3 opening the catheter flange coming from a side approach.

FIG. 6B is a side elevational view of the device of FIG. 3 opening the catheter flange coming from a side approach.

### DESCRIPTION OF THE SELECTED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any alterations, modifications, and further applications of the principles of the present invention as illustrated being contemplated as would normally occur to one skilled in the art to which the invention relates.

Generally speaking, a device for opening a substantially flat external flange of an indwelling catheter is described. The device is a one-piece elongated member having configured ends. The device is formed by bending the elongated member so that one configured end is positioned slightly above the other configured end by passing the second configured end through an aperture formed inside of the member. In addition, generally speaking, an apparatus comprising at least the opening device and an indwelling catheter having a flat external flange is described. The device is used to maintain the low profile of the external flange of the indwelling catheter and to prevent damage to the skin or body tissue surrounding the catheter flange.

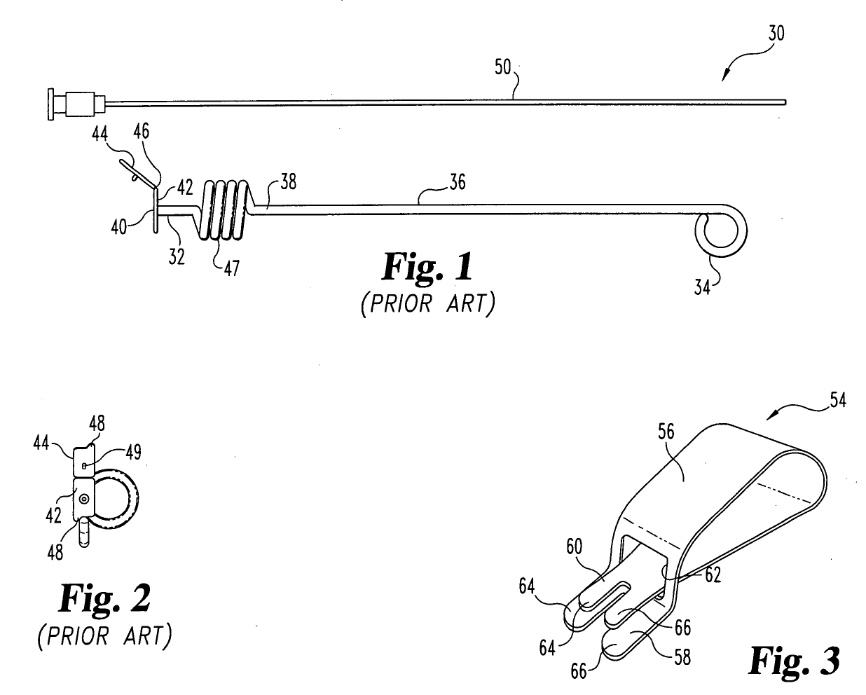

Referring now to the drawings, FIG. 1 illustrates an indwelling catheter 30. Indwelling catheters of this type are known in the prior art. For example, reference to U.S. Pat. No. 5,941,823 to Chait, U.S. Pat. No. 5,727,555 to Chait or U.S. Pat. No. 6,223,070 to Chait describe indwelling catheters with flat external flanges. It should be appreciated by those skilled in the art; however, that many other types of catheters other than those listed in the aforementioned patents can be opened by the opening device described hereinbelow with reference to FIG. 3. The indwelling catheter 30 has a proximal end 32, a distal end 34 and elongate body 36 between the proximal end 32 and the distal end 34. The elongate body 36 defines a lumen 38 therein. The elongate body 36 carries fluids between the proximal end 32 and the distal end 34 when the indwelling catheter 30 lies inside the patient's body. The catheter 30 also has an external flange 40. The external flange 40 is substantially flat. The external flange 40 is to close off the access area of the catheter 30 when not in use. The flange 40 comprises a base 42 which is a plate-like member and a cover 44 which is also a plate-like member that are coupled together at hinge 46. Referring to FIG. 2, the cover 44 and the base 42 also have tabs 48 at one end. The cover 44 has a bulged pin 49 that fits into the inner diameter of the catheter 30 to couple the cover 44 to the base 42 and thereby close the external flange 40. The bulged pin 49 ensures a tight close of the external flange 40, however, this feature adds to the difficulty in opening and closing the external flange 40. In addition, the catheter 30 also includes coils 47 that extend when a stiffener 50 is placed into the catheter 30. During insertion of the catheter 30 into the body, the coils 47 retract upon removal of the stiffener 50 after the catheter 30 has been sufficiently inserted. The coils 47 insure that the catheter 30 stays inside of the body once inserted. The external flange 40 is normally closed during use and only opened in order to remove the catheter or to provide a passage for fluids to either enter or exit the body.

Referring now to FIG. 3, a preferred embodiment of opening device 54 is illustrated. The opening device 54 is formed of any material readily apparent to those skilled in the art in differing embodiments. In some embodiments, the material is a resilient material that will return to its original shape after the application of compressive forces. For instance, rubber, a plastic with a low level of brittleness, or composite material can be used in some embodiments. In addition, in the illustrated embodiment, the opening device 54 is constructed from a substantially flat elongate member. Those skilled in the art, however, will recognize that the opening device could be constructed from other configurations, so long as the device 54 is constructed to slide inside and separate the external flange 40 of the indwelling catheter 30.

The opening device 54 has a body portion 56 that connects the first configured end portion 58 and the second configured end portion 60. The second configured end portion 60 is positioned above the first configured end portion 58 by bending the body portion 56 back around itself and passing the second configured end portion 60 through an aperture 62 formed in body portion 56. This places the first configured end portion 58 in a proximate parallel plane to the second configured end portion 60. Thus, when compression is applied, the first configured end portion 58 and the second configured end portion 60 move away from one another, and when compression is removed, the first configured end portion 58 and the second configured end portion 60 move toward one another.

The first configured end portion 58 and second configured end portion 60 each have a first extending portion 64 and a second extending portion 66. The extending portions 64, 66 define a general fork shape for the configured end portions 58, 60. It is contemplated, however, that in other embodiments, other configurations of extending portions or even no extending portions at all are contemplated. In some embodiments, the extending portions could be triangular shaped, rectangular shaped, circular shaped or even spherical. In addition, other extending portion configurations readily apparent to those skilled in the art are contemplated. While the embodiment illustrated in FIG. 3 has a body portion 56 that has been bent back upon itself, other alternate ways of creating an opening device 54 are contemplated. The only requirement of the opening device 54 is that the device is constructed and arranged to slide between and separate the plates of the external flange 40.

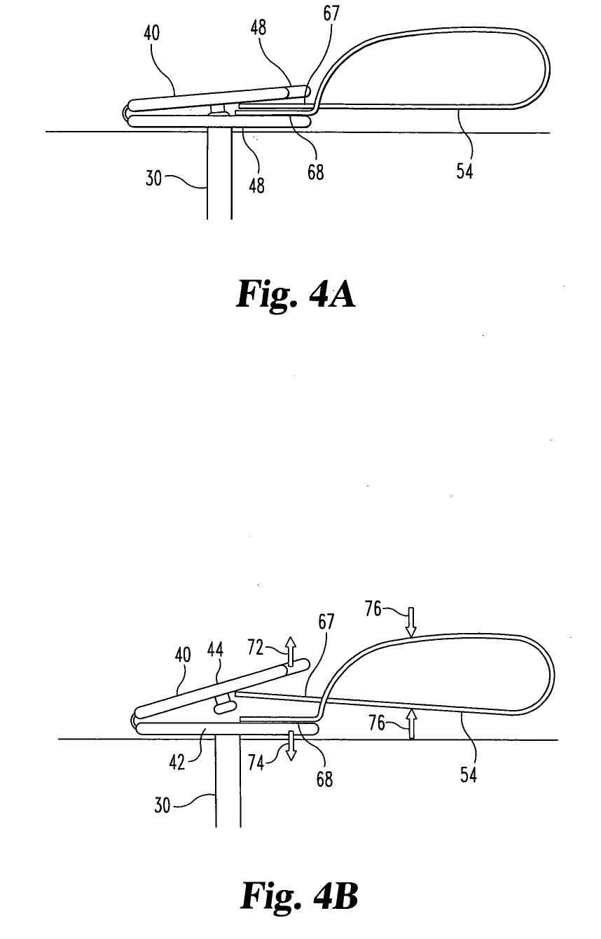

Referring now to FIG. 4A, the device illustrated in FIG. 3 is shown from a side elevational view. The device 54 has an upwardly facing surface 67 and a downwardly facing surface 68. The upwardly facing surface 67 is a top surface of the second configured end portion 60 and the downwardly facing surface 68 is a bottom surface of the first configured end portion 58. These surfaces 67, 68 are slid between the plates to contact the external flange 40 at tabs 48. Before the opening device 54 is placed into the external flange 40, the user commonly will manually press down gently to steady the indwelling catheter 30 before the device 54 is put into place.

FIG. 4B illustrates the opening device 54 opening the external flange 40. The upwardly facing surface 67 places an upward force 72 upon the cover 44 of the external flange while the downwardly facing surface 68 places a downward force 74 to the base 42 of the external flange 40. The external flange 40 is therefore opened without lifting the external flange 40 or causing any damage to the surrounding skin areas. Applying a compressive force 76 to the opening device 54, either manually or using some other medical device, creates the forces 72 and 74.

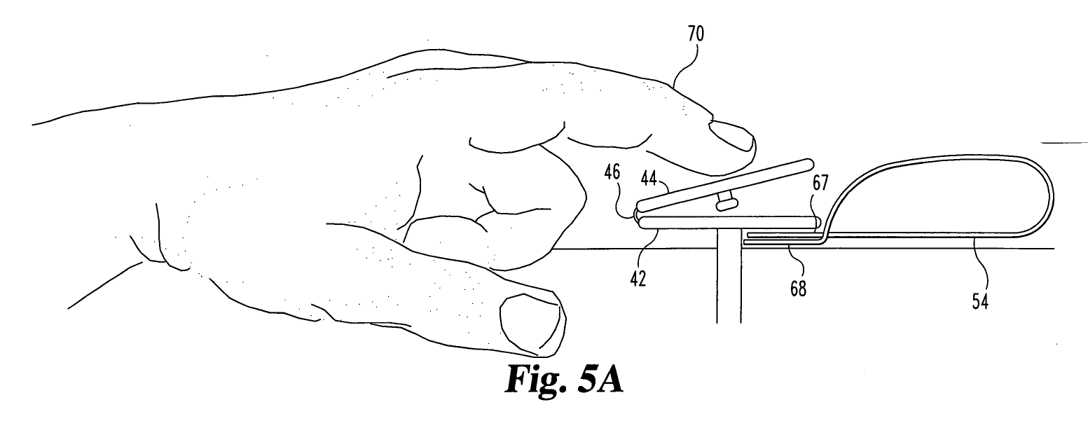

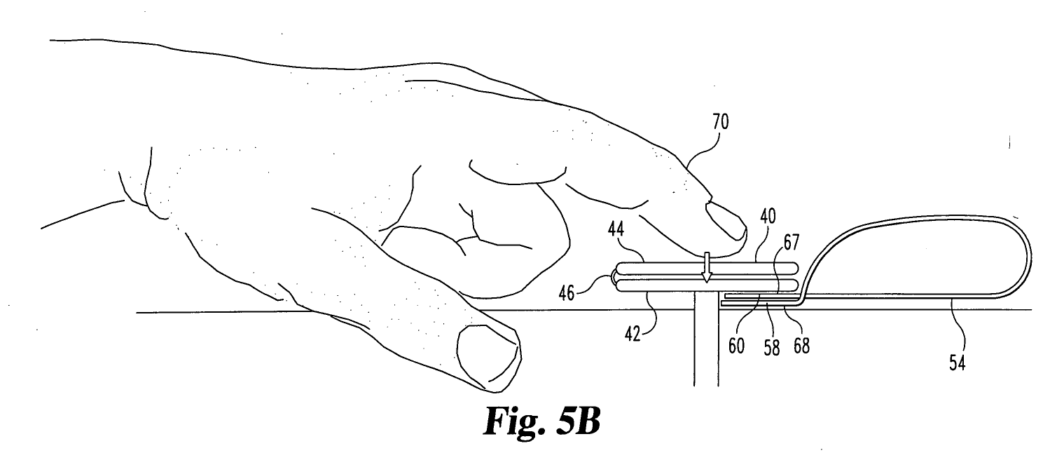

Referring now to FIG. 5A, the device illustrated in FIG. 3 is slid underneath the base 42 of the external flange 40. After the opening device 54 is placed under the external flange 40, the user commonly will press down gently using a finger 70 to steady the indwelling catheter 30.

FIG. 5B illustrates the closing of the external flange 40 of the device 54. The configured ends 58, 60 of the device 54 have been placed underneath the base 42 of the external flange 40 and the user is holding the cover steady with the finger 70. The external flange 40 is held steady with respect to the skin surface by the configured ends 58, 60 of the device 54. The finger 70 therefore can easily close the external flange 40 without substantial stress on the skin surrounding the external flange 40. Damage to the skin around the external flange 40 is thereby lessened. Moreover, a very low profile is easily maintained.

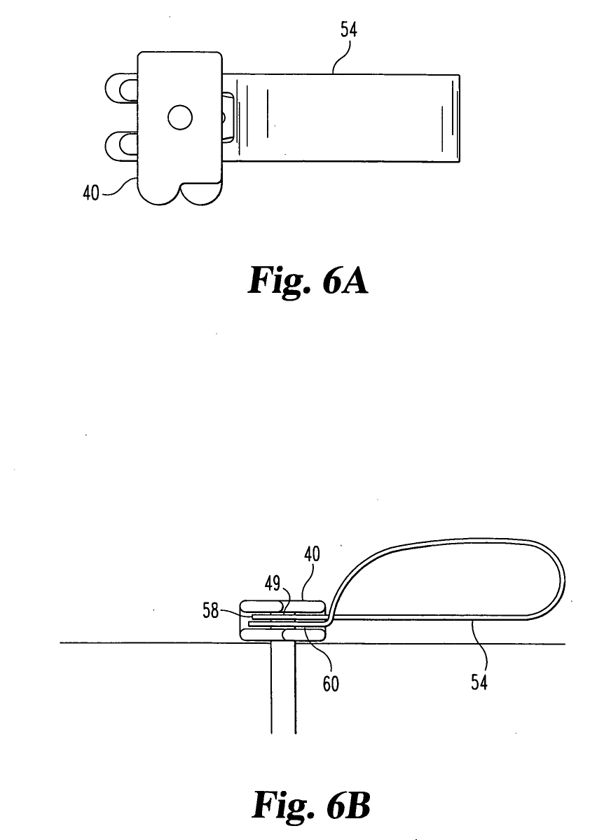

FIG. 6A illustrates the device 54 opening the flange 40 from a top plan view. Moreover, the FIG. 6A illustrates that the flange 40 can be opened from the side of flange 40. Thus, the opening of the tabbed end of the flange 40 is not required. Similarly, FIG. 6B illustrates the device 54 entering the flange 40 from the side. This figure illustrates how the configured ends 58, 60 of the device include clearance for the bulged pin 49 and therefore allow a side entry into the flange 40. Therefore, the device 54 can open the flange 40 from almost any direction increasing its usefulness.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. A device for opening the access area of a catheter comprising:
a substantially flat elongate member comprising:
a first configured end portion;
a second configured end portion; and
a body portion between said configured end portions, said body portion defining an aperture
wherein said body portion is formed so as to position said second configured end portion above said first configured end portion by passing said second configured end portion through said aperture in said body portion.

2. The device of claim 1, wherein said configured end portions are forked.

3. The device of claim 1, wherein said configured end portions lie in proximate parallel planes.

4. The device of claim 1, wherein said device is formed of a resilient material.

5. The device of claim 1, wherein said configured end portions define a first extending portion and a second extending portion.

6. The device of claim 5, wherein each said extending portion is rectangularly shaped.

7. The device of claim 5, wherein each said extending portion is triangularly shaped.

8. An apparatus comprising:
a catheter having a substantially flat external flange; and
an opening device comprising:
a first configured end portion;
a second configured end portion; and
a body portion between said configured end portions, said body portion defining an aperture,
wherein said body portion is formed so as to position said second configured end portion above said first configured end portion by passing said second configured end portion through said aperture,
wherein said opening device opens said external flange of said catheter.

9. A medical apparatus for a human body comprising:
a catheter comprising:
an elongate body having a proximal end, a distal end and an interior lumen therethrough;
an external flange having a substantially flat base, a substantially flat cover and a hinge coupling the cover to the base; and
a plurality of helical coils formed in the body between the proximal and distal ends, wherein a stiffener is placed into the lumen to straighten the coils to enable insertion into the body and said coils reform upon removal of said stiffener to enable retention of said catheter in said body; and
a device constructed and arranged to slide between and physically separate the base and the cover of the external flange.

10. The medical apparatus of claim 9 wherein said device physically separates said base and said cover using an upwardly facing surface and a downwardly facing surface that separate upon the application of a compressive force to said device.

11. The medical apparatus of claim 9 wherein said device includes extending portions to slide between said base and said cover of said external flange.

12. The medical apparatus of claim 9 wherein said device further includes a second configured end and a first configured end.

13. The medical apparatus of claim 12 wherein the first configured end is placed over the second configured end by passing said end through an aperture defined in said device.

14. A method for using an opening device having at least two configured portions to open a catheter having a substantially flat external flange formed having two substantially flat plates, the method comprising:
inserting said configured portions between said substantially flat plates; and
compressing said opening device to apply forces to said substantially flat plates using said configured portions.
